(19) United States
(12) Patent Application Publication
(10) Pub. No.: US 2017/0304021 A1
(43) Pub. Date: Oct. 26, 2017

(54) SURGICAL ROBOTIC ARM SUPPORT SYSTEMS AND METHODS OF USE

(71) Applicant: Covidien LP, Mansfield, MA (US)

(72) Inventor: Peter Hathaway, Lebanon, CT (US)

(73) Assignee: Covidien LP, Mansfield, MA (US)

(21) Appl. No.: 15/512,583

(22) PCT Filed: Sep. 16, 2015
(86) PCT No.: PCT/US15/50349

§ 371 Date: Mar. 20, 2017

Related U.S. Application Data
(60) Provisional application No. 62/054,025, filed on Sep. 23, 2014.

(51) Int. Cl. A61B 90/50; A61B 34/30; A61G 13/10; B65H 75/38

(52) U.S. Cl. CPC A61B 90/50; B65H 75/38; A61B 34/30; A61G 13/101

(57) ABSTRACT

A surgical robotic arm support system includes a rail and one or more mounting members configured to be coupled to the rail at a selected one of a plurality of discrete robotic arm mounting positions. Each mounting member is configured to support a surgical robotic arm. Distances from the discrete mounting positions to a surgical site access point on a patient may be calculated based on the mounting position locations and a patient dimension and/or a placement position of the patient on a surgical table. An optimal mounting position for each of an optimal number of the surgical robotic arms for a selected surgical procedure may be identified from the calculated distances.

### CROSS-REFERENCE TO RELATED APPLICATIONS

This application is a U.S. National Stage Application filed under 35 U.S.C. §371(a) of International Patent Application No. PCT/US2015/050349, filed Sep. 16, 2015, which claims the benefit of and priority to U.S. Provisional Patent Application No. 62/054,025, filed Sep. 23, 2014, the entire disclosure of which is incorporated by reference herein.

### BACKGROUND

Robotic surgical systems have been used in minimally invasive medical procedures. Some robotic surgical systems included a console supporting surgical robotic arms that manipulated respective surgical instruments and their end effectors (for example, forceps or grasping tools) attached to the robotic arms. A robotic arm provided mechanical power to the surgical instrument for its operation and movement through an instrument drive unit coupled to the surgical instrument.

Before a surgery started, the surgical robotic arms were manually positioned so that the surgical instruments that entered the patient's body were generally aligned with trocars in the patient through which the instruments were inserted. The manually positioning and adjustment process was time consuming and involved varying degrees of trial and error. There is a need for a more efficient process for positioning surgical robotic arms that reduces the overall setup time in the operating room.

### SUMMARY

Surgical robotic arm setup times may be reduced by detachably mating each of the robotic arms to a pre-selected one of a series of uniquely identified fixed mating points on a fixed object such as a surgical table. Each of the mating points may be fixedly positioned at predetermined distances from each other and/or from a reference point on the surgical table. Each of the mating points may be uniquely identified with different numbers, images, colors, symbols or identifiers. Patient-specific information (e.g. the type of surgical procedure being performed or a size, sex, or placement of the patient) may be analyzed based on robotic arm placement optimization criteria to pre-select those mating points to which the robotic arms should be attached. The identifiers of the pre-selected mating points may be outputted so that the robotic arms can be quickly mated to the respective pre-selected mating point without having to go through a time consuming trial and error process for manually positioning the arms.

A surgical robotic arm support system may include a rail and at least one mounting member. The rail is configured to be coupled to a surgical table and includes a plurality of discrete robotic arm mounting positions. The mounting member may be attached to or integrated into the robotic arm and may be configured to detachably mate the robotic arm to the rail at only a selected one of the plurality of discrete robotic arm mounting positions. Each mounting member may be configured to support a surgical robotic arm.

Each of the plurality of discrete robotic arm mounting positions may have a different identifier associated therewith. The surgical robotic arm support system may further include a processor. The processor may be configured to identify a target mounting position for each surgical robotic arm relative to the surgical table. The processor may be configured to indicate the identified target mounting position of each surgical robotic arm by displaying the specific identifier corresponding to the identified target mounting position.

It is envisioned that each mounting member may include a channel configured for slidable receipt of a respective surgical robotic arm.

In some embodiments, the mounting member may be configured to be slidingly coupled to the rail.

In some aspects, the surgical robotic arm support system may further include a spool configured to be coupled to a fixed surface, e.g., a ceiling. The spool may include a retractable tether having an end configured to be attached to the surgical robotic arm.

It is contemplated that the surgical table may have a long side and a short side.

The rail may be mounted to the long side.

In embodiments, the surgical robotic arm support system may further include a coil disposed about the surgical robotic arm configured to cool the surgical robotic arm as a cooling medium passes through the coil.

According to another aspect of the present disclosure, another embodiment of a surgical robotic arm support system is provided. The surgical robotic arm support system includes a surgical table and at least one mounting member. The surgical table is for supporting a patient thereon. The surgical table includes a rail having a plurality of discrete robotic arm mounting positions. The mounting member is configured to be coupled to the rail at a selected one of the plurality of discrete robotic arm mounting positions. Each mounting member is configured to support a surgical robotic arm.

According to yet another aspect of the present disclosure, a method of mounting surgical robotic arms to a surgical table is provided. The method includes providing a surgical table having a plurality of discrete mounting positions for surgical robotic arms. A target mounting position of a surgical robotic arm relative to the surgical table is determined. A mounting member is coupled to the determined target mounting position. The surgical robotic arm is coupled to the mounting member.

In embodiments, the method may further include displaying discrete indicia that corresponds to the determined target mounting position of the surgical robotic arm. The mounting member may be coupled to the determined target mounting position based on matching the discrete indicia associated with a respective discrete mounting position with the displayed discrete indicia of the target mounting position.

In some aspects, the target mounting position of the surgical robotic arm relative to the surgical table may be determined by providing surgical parameters to a virtual surgical procedure simulator. The surgical parameters may include dimensions of a patient, a target tissue area of the patient, dimensions of the surgical robotic arm, and dimensions of the surgical table. The virtual surgical procedure simulator may provide suggested target mounting positions for the surgical robotic arms relative to the surgical table. The surgical parameters may be entered into the simulator via a user interface.

It is contemplated that the mounting member may be coupled to the target mounting position by sliding the mounting member along a rail of the surgical table to the target mounting position.

It is envisioned that the surgical robotic arm may be coupled to the mounting member by inserting the surgical robotic arm into a channel defined in the mounting member. The surgical robotic arm may be secured in the channel.

In embodiments, the method may further include coupling a spool to a ceiling of an operating room and attaching an end of a retractable tether of the spool to the surgical robotic arm.

In some aspects, the method may further include passing a cooling medium through a coil disposed about the surgical robotic arm to cool the surgical robotic arm.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about +or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a robotic surgical system, including a surgical robotic arm, of the present disclosure;

FIG. 2 is a perspective view of the surgical robotic arm of the robotic surgical system of FIG. 1 illustrating a surgical instrument attached thereto;

FIG. 3 is a side, schematic view of a surgical robotic arm support system according to the present disclosure illustrating a mounting member mounted to a surgical table;

FIG. 4 is a schematic view of a virtual surgical procedure simulator of the surgical robotic arm support system of FIG. 3;

FIG. 5 is a top, perspective view of the surgical robotic arm support system of FIG. 3 and the surgical robotic arm of FIG. 2 illustrating the surgical robotic arm tethered to a ceiling of an operating room; and

FIG. 6 shows an exemplary method for optimizing robotic arm placement.

### DETAILED DESCRIPTION

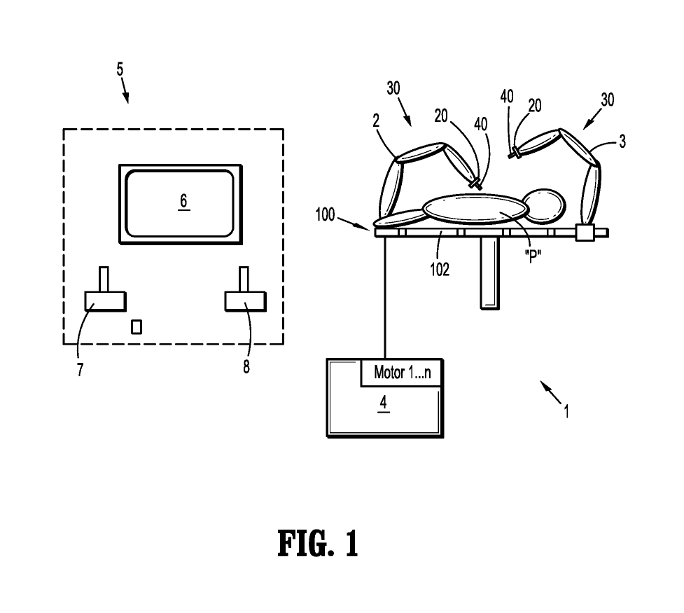

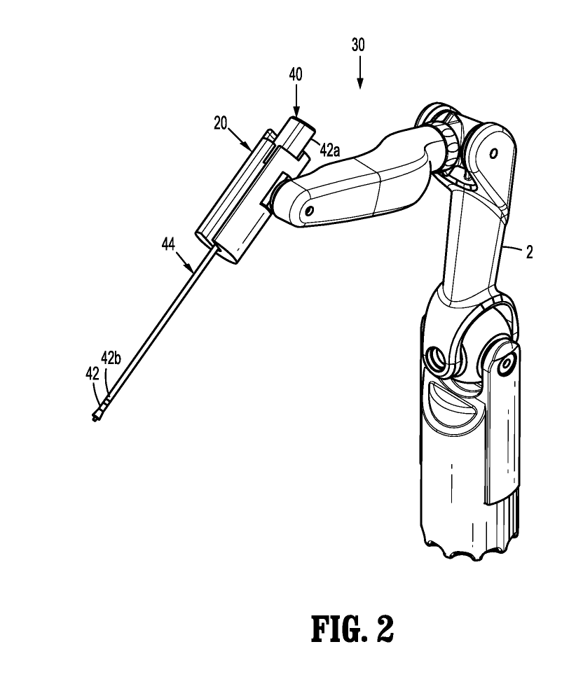

Referring initially to FIGS. 1 and 2, a surgical system, such as, for example, a robotic surgical system 1, generally includes a plurality of surgical robotic arms 2, 3; a control device 4; an operating console 5 coupled with control device 4, and a surgical robotic arm support system 100. Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Each of the robotic arms 2, 3 may be composed of a plurality of members, which are connected to one another through joints. Robotic surgical system 1 also includes an instrument drive unit 20 connected to distal ends of each of robotic arms 2, 3. A surgical instrument 40 supporting an end effector 42 may be attached to instrument drive unit 20, in accordance with any method known by one having skill in the art.

Robotic arms 2, 3 may be driven by electric drives (not shown) that are connected to control device 4. Control device 4 (e.g., a computer) is set up to activate the drives, in particular by means of a computer program, in such a way that robotic arms 2, 3, their instrument drive units 20, and thus the surgical instrument 40 (including end effector 42) execute a desired movement according to a movement defined by means of manual input devices 7, 8. Control device 4 may also be set up in such a way that it regulates the movement of robotic arms 2, 3 and/or movement of the drives (not shown).

Robotic surgical system 1 is configured for use on a patient “P” lying on a patient table, such as, for example, a surgical table 102 to be treated in a minimally invasive manner by means of an end effector. Robotic surgical system 1 may also include more than two robotic arms 2, 3, the additional robotic arms likewise being connected to control device 4 and being telemanipulatable by means of operating console 5. A surgical instrument, for example, surgical instrument 40 (including end effector 42), may also be attached to the additional robotic arm.

For a detailed discussion of the construction and operation of robotic surgical system 1, reference may be made to U.S. Patent Publication No. 2012/0116416, filed on Nov. 3, 2011, entitled “Medical Workstation,” the entire content of which is incorporated herein by reference.

Control device 4 may control a plurality of motors (Motor 1 . . . n) with each motor configured to drive a pushing or a pulling of a cable (not shown) extending between end effector 42 of surgical instrument 40 and a respective driven member (not shown) of surgical instrument 40. In use, as the cables are pushed or pulled relative to end effector 42, the cables effect operation and/or movement of each end effector 42 of surgical instrument 40. It is contemplated that control device 4 coordinates the activation of the various motors (Motor 1 . . . n) to coordinate a pushing or a pulling motion of a respective cable in order to coordinate an operation and/or movement of a respective end effector 42. In embodiments, each motor can be configured to actuate a drive rod or a lever arm to effect operation and/or movement of each end effector of surgical instrument 40.

With specific reference to FIG. 2, robotic surgical system 1 includes a surgical assembly 30, which includes a robotic arm 2, an instrument drive unit 20 connected to robotic arm 2, and a surgical instrument 40 coupled with or to instrument drive unit 20. Instrument drive unit 20 is configured for driving an actuation of end effector 42 of surgical instrument 40 and to operatively support surgical instrument 40 therein. Instrument drive unit 20 transfers power and actuation forces from motors to surgical instrument 40 to ultimately drive movement of the cables that are attached to end effector 42. Instrument drive unit 20 includes a plurality of driving members (not shown) attached to a respective motor such that the drive members are independently rotatable with respect to one another.

Surgical instrument 40 generally has a proximal end portion 42a configured to be engaged with instrument drive unit 20 and a distal end portion 42b having end effector 42 extending therefrom. Surgical instrument 40 further includes an elongate body or tube 44. End effector 42 extends distally from distal end 42b of elongate body 44 and is configured for performing a plurality of surgical functions.

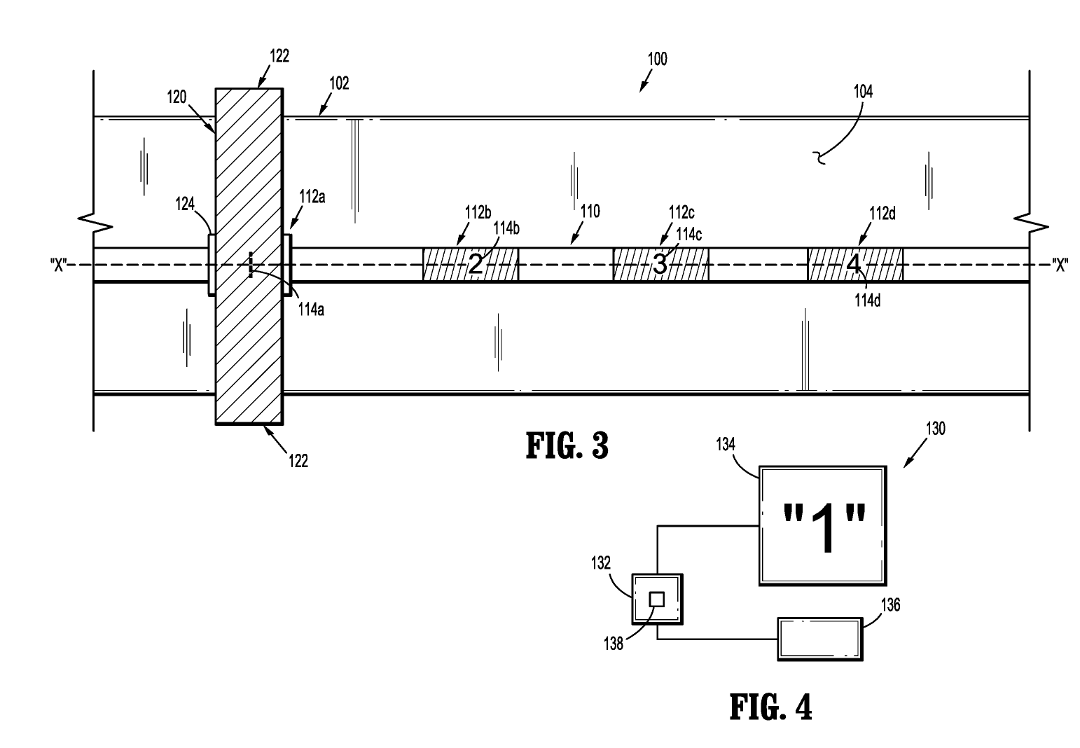

Turning to FIG. 3, robotic surgical system 1 also includes a surgical robotic arm support system 100 for selectively positioning surgical assembly 30 including robotic arms 2, 3 relative to patient “P” and for supporting surgical robotic arms 2, 3 in the selected position. Surgical robotic arm support system 100 may include a support member, such as, for example, a rail 110, coupled to surgical table 102, and a mounting member 120 configured to be coupled to rail 110.

Surgical table 102 is configured for supporting a patient thereon. Surgical table 102 defines a longitudinal axis “X” and may have longitudinally extending lateral sides 104. One or more rails 110 may be mounted to one or more of the sides of the surgical table 102. The rails 110 may be removably coupled to side 104 of surgical table 102, fixedly attached to the table 102 or integrally formed therewith. In embodiments, surgical table 102 may include one or more rails mounted to one or more lateral sides or other surfaces of surgical table 102. In some instances, instead of a rail 110 being attached to the table 102, a series of discrete mounting units, each having at least one robotic arm mounting position 112 thereon, may be removably or fixedly attached to the surgical table 102 at predetermined distances from each other and/or from a fixed point (e.g. a preselected corner of the surgical table 102).

Rail 110 includes a plurality of discrete robotic arm mounting positions 112a, 112b, 112c, 112d longitudinally spaced from one another along longitudinal axis “X” of surgical table 102. Each discrete mounting position 112a, 112b, 112c, 112d occupies an area or zone along side 104 of surgical table 102 in which one surgical robotic arm 2 or 3 (FIG. 2) may be selectively positioned prior to and/or during a surgical procedure performed on patient “P” (FIG. 1). In embodiments, each discrete mounting position 112a, 112b, 112c, 112d is located directly on side 104 of surgical bed 102. In one embodiment, discrete mounting positions 112a, 112b, 112c, 112d are defined by a suitably dimensioned groove or notch formed in rail 110, with a corresponding mating tab on the mounting member 120 for matingly attaching the mounting member 120 to the mounting position 112. In some instances, a tab may be provided on the rail 110 and the groove or notch may be formed in the mounting member 120. Other mating arrangements for matingly attaching the mounting member 120 to a discrete mounting position 112 may be used in other instances.

Surgical robotic arm support system 100 may include more than one mounting member 120. Each mounting member 120 is configured to mount a respective surgical robotic arm 2, 3 to surgical table 102 at a selected discrete mounting position 112a, 112b, 112c, or 112d. Each mounting member 120 may be in the form of a tube that defines a channel 122 therethrough configured for slidable receipt of a base of surgical robotic arm 2, 3. In some instances, mounting member 120 may be integrated into or part of a surgical robotic arm 2, 3. Each mounting member 120 may include one or more attachments 124 configured to connect the mounting member 120 to rail 110 of surgical table 102. Attachment 124 may sit in a respective groove, notch, protrusion, tab, or the like formed in rail 110 that defines respective a mounting position 112a, 112b, 112c, 112d and mates to a respective tab, groove, protrusion, notch, or the like in the attachment 124. Attachments 124 may be slidingly coupled to rail 110 such that the mounting member 120 may be moved, slid, or, translated longitudinally along rail 110 into a selected discrete mounting position 112a, 112b, 112c, 112d along side 104 of surgical table 102. In embodiments, mounting members 120 may be directly connected to a mounting position 112 at the side 104 of surgical table 102 without the use of rail 110, for example, via various fastening engagements, such as, for example, snap-fit engagement, frictional engagement, adhesives, and/or various fasteners.

In some embodiments, attachments 124 may be in the form of various fasteners, such as, for example, c-clips, brackets, straps, buckles, magnets, suction cups, or the like. In some embodiments, mounting members 120 are fixedly connected with rail 110 in respective discrete mounting positions 112a, 112b, 112c, 112d. The attachments 124 may be detachable from mounting members 120.

With continued reference to FIG. 3, each robotic arm mounting position 112a, 112b, 112c, 112d has discrete indicia 114a, 114b, 114c, 114d associated therewith to assist a clinician in identifying each discrete mounting position 112a, 112b, 112c, 112d, as described in greater detail below. In the embodiment illustrated in FIG. 3, discrete indicia 114a, 114b, 114c, 114d are in the form of Arabic numerals (i.e., 1, 2, 3, 4, etc.) in ascending order from left to right along rail 110. In embodiments, discrete indicia 114a, 114b, 114c, 114d may be in various forms, such as, for example, discrete colors, letters, symbols, or other distinctive markings, labels, or stamps unique to each discrete mounting position 112a, 112b, 112c, 112d that aid in visually distinguishing and identifying mounting positions 112a, 112b, 112c, 112d. In embodiments, discrete indicia 114a, 114b, 114c, 114d may be displayed directly on surgical bed 102 or on respective mounting members 120.

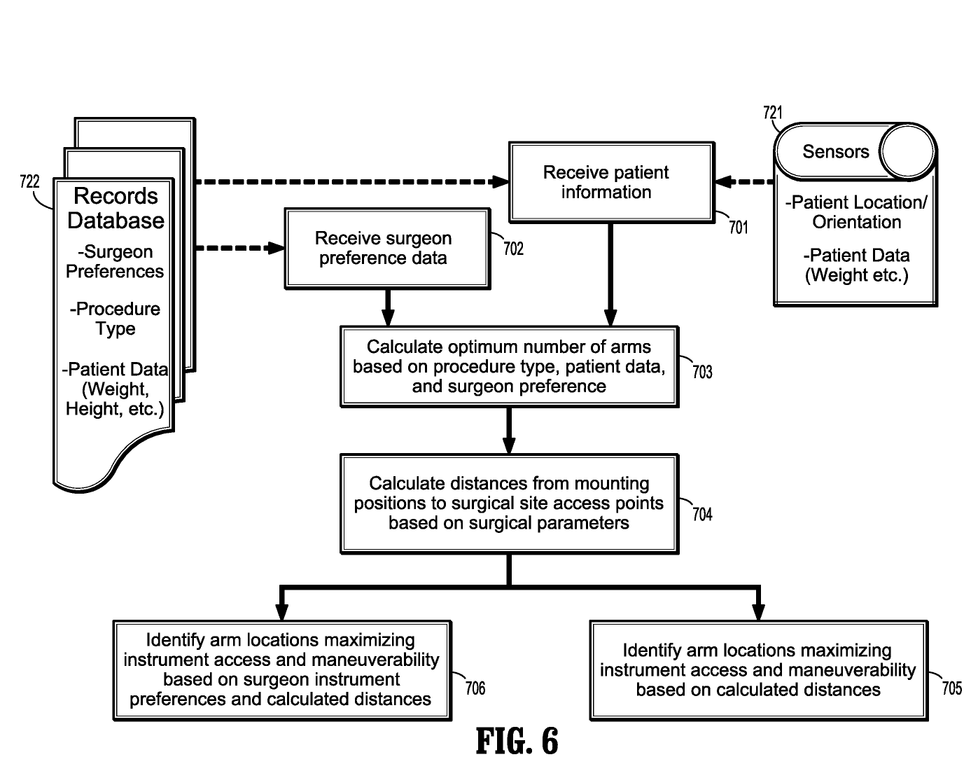

With reference to FIG. 4, surgical robotic arm support system 100 further includes an optimization simulator 130 including a processor 132, a display 134 in communication with processor 132, and a user interface 136. An exemplary method for optimizing robotic arm placement using optimization simulator 130 is shown in FIG. 6. Processor 132 is capable of executing a series of instructions, algorithms, or protocols that are stored in a memory 138, e.g., a storage device and/or external device (not shown). User interface 136 is communicatively coupled to processor 132 so that a user, for example, a clinician, can input information for processor 132 to process.

Memory 138 may store the location of each possible target mounting position for surgical robotic arms 2, 3; the discrete indicia associated with each stored possible target mounting position; and optimization criteria for optimizing the placement of the robotic arms 2, 3 in different instances. Each discrete indicia and each location stored in memory 138 of processor 132 may correspond to one of the discrete indicia 114a, 114b, 114c, 114d associated with the location of respective discrete mounting positions 112a, 112b, 112c, 112d. Processor 132 may be configured to use the optimization criteria in memory 138 to identify the optimum target mounting positions for each surgical robotic arm 2, 3 relative to surgical table 102 in a particular instance and output the identified target mounting position (one or more of mounting positions 112a, 112b, 112c, 112d) by displaying the discrete indicia 114a, 114b, 114c, and/or 114d corresponding to the identified target mounting position 112a, 112b, 112c, and/or 112d on display 134.

In some instances, the optimization simulator 130 may use additional patient information as part of the optimization criteria to identify the optimum location for each of the robotic arms 2, 3. This additional patient information may include information about the type of surgical procedure, information about a physical characteristic of the patient (e.g. size, weight, body mass index, height, sex, etc.), and/or information about a placement of the patient on the surgical table 102 (e.g. an orientation of the patient on the table 102, an identification of a mounting position 112 closest of a particular part of the patient, a location of the patient with respect to a specific mounting position 112 or part of the table 102, etc.).

In box 701, this patient information may be received at the processor 132 from a records database 722 and/or one or more sensors 721 communicatively coupled to the processor 132. Patient information may also be entered manually or be automatically retrieved. Some information may be automatically retrieved through an inference with the patient record database 722 and/or through the sensors 721 affixed to the surgical table 102, rail 110, and/or mounting positions 112. The sensors 721 may include pressure sensors, position sensitive detectors, proximity sensors, imaging sensors, and other sensors that are able to detect placement information about the patient on the surgical table 102 (e.g. an orientation of the patient on the table 102, an identification of a mounting position 112 closest of a particular part of the patient, a distance of the patient with respect to a specific mounting position 112 or part of the table 102, a location of an object or body part of the patient etc.) or detect a physical characteristic of the patient (e.g. weight, height, sex, etc.).

In box 703, the received patient information may be inputted into a formula or compared against benchmarked data for the selected surgical procedure to determine optimal number of robotic arms 2, 3 for the selected surgical procedure in view of the identified physical characteristics and placement of the patient on the surgical table 102. In some instances, for example, for a particular procedure, the procedure may be most efficiently performed on a short, thin patient with three robotic arms positioned relatively close to the surgical site. The same procedure may be most efficiently performed on a tall, slim patient with three robotic arms spread further apart. The same procedure may also be most efficiently performed on an obese patient with four robotic arms located at different positions and spread further apart. Different target mounting position 112a, 112b, 112c, and/or 112d may also be selected depending on where on the table 102 the patient is positioned.

In some instances, the relative range of motion and degrees of movement of robotic arms 2, 3 may also be considered when identifying the optimal placement of robotic arms 2, 3 to mounting positions 112a, 112b, 112c, 112d. For example, if the abdominal area of patient “P” positioned in the middle of surgical table 102 is to be operated on, it can be appreciated that it would be more suitable to mount surgical robotic arms 2, 3 as close to middle of surgical table 102 as possible, to be closer to the abdominal area of patient “P,” given the limited reach of surgical robotic arms 2, 3.

Accordingly, to determine the most advantageous mounting positions 112a, 112b, 112c, 112d on surgical table 102 for surgical robotic arms 2, 3, one or more surgical parameters may be determined from the above mentioned patient information 701. Surgical parameters may include, for example, dimensions of patient “P,” a target tissue area of patient “P,” dimensions of robotic arms 2, 3, the dimensions of surgical table 102, distances from the target tissue area to a surgical instrument affixed to a robotic arm 2, 3, and so on. One or more of these surgical parameters may be manually entered, retrieved from a database or memory, or may be computed from additional patient or surgical procedure information, such as a characteristic of the patient, surgical procedure information, patient placement information, and/or surgical table information. In one embodiment, the determined dimensions of patient “P,” the determined dimensions of the target tissue area of patient “P,” the dimensions of surgical table 102, the dimensions of robotic arms 2, 3, and the dimensions of surgical assemblies 30 are entered into optimization simulator 130 via user interface 136.

In box 704, the surgical parameters may be determined and the distances from mounting positions to surgical site access points may be calculated based on the surgical parameters. The surgical parameters may be provided to simulator 130 via various methods. For example, in embodiments, surgical parameters may be pre-programmed into simulator 130, obtained from one or more sensors in real time, or automatically uploaded into simulator 130 from a device such as a robotic arm 2, 3 (e.g., wirelessly uploaded) when the particular surgical robotic arm 2, 3 is brought into the operating room, placed in mounting member 120, or otherwise connected to a communications network enable the uploading of data.

In box 705, the processor 132 may identify the optimum arm locations for each of the robotic arm 2, 3 that maximizing instrument access and maneuverability based on stored positional information of the discrete mounting positions 112 and the distances calculated in box 704 using an optimization algorithm based on benchmarked data. The algorithm and/or the benchmarked data may be stored in memory 138. The identified optimum number of robotic arms 2, 3, and the identified optimum placement positions for each of the surgical robotic arms 2, 3 may then be outputted to the user. In some instances, only one suggested target mounting position may be outputted but in other instances more than one mounting position may be outputted, such as, for example a list of alternative next-best mounting positions.

In some instances, preferences of a surgeon performing the surgical procedure may be stored in a database such as records database 722. In box 702, this preference data for the surgeon performing the procedure may be retrieved from the database 722 and received at the simulator 130 and/or processor 132. The stored surgeon preferences may include a preferred number of robotic arms used by the surgeon for the surgery, preferred instruments used by the surgeon during the surgery, preferred placement of ports used to provide access for the surgical instruments on the robotic arm 2, 3 to the surgical site, and/or other preferences of the surgeon.

If the surgeon prefers a particular number of robotic arms 2, 3 then the simulator 130 may select the surgeon preferred number of robotic arms 2, 3 as the calculated optimum number of arms in box 703 for the particular surgical procedure. If the surgeon has other preferences, such as a preferred location of trocars or ports providing surgical instrument access to the surgical site, then these preferred locations may be used instead as part of the surgical parameter determinations and distance calculations in box 704. If the surgeon prefers a specific subset of surgical instruments for the particular surgical procedure then in box 706, the discrete arm mounting locations that maximize the access and maneuverability of the particular subset of surgeon preferred instruments at the surgical site may be calculated and outputted instead. In box 706, the simulator 130 may also identify specific robotic arms 2, 3 that each of the preferred instruments should be attached to individually maximize access and maneuverability of each instrument at the surgical site.

Upon calculating the suggested target mounting positions of each robotic arm 2, 3 on specific mounting positions 112, processor 132 may output the suggested target mounting position of each surgical robotic arm 2, 3 by displaying, on display 134, the stored discrete indicia (e.g., Arabic numeral 1 as shown in FIG. 4) corresponding to the calculated suggested target mounting position. As such, a clinician is able to visually match the displayed discrete indicia (virtual indicia) with the discrete indicia 114a, 114b, 114c, 114d (real indicia) associated with each discrete mounting position 112a, 112b, 112c, 112d on surgical table 102. Mounting member 120 is then coupled, via translation, sliding, or other movement along rail 110, to the discrete mounting position along surgical table 102 that has discrete indicia matching the displayed discrete indicia (i.e., a “1” is displayed on display 134 indicating mounting member 120 is to be positioned at discrete mounting position 112a, which has discrete indicia “1” associated therewith).

In embodiments, upon calculating the suggested target mounting positions, processor 132 may cause mounting members 120 to move to the suggested mounting positions automatically via motors or some other driving means (not shown) without the aid of a clinician. Encoders in the motors or driving means or other sensors may be used to verify the proper positioning of the robotic arms and/or their mounting members 120 to the calculated target positions.

With continued reference to FIGS. 3 and 4, surgical robotic arm 2 (FIG. 1) is inserted into channel 122 of mounting member 120 and secured therein. As such, surgical robotic arm 2 is coupled to rail 110 at the target mounting position 112a determined by optimizer simulator 130. In embodiments, surgical robotic arm 2 may be coupled to mounting member 120 before or after mounting member 120 is coupled to the target mounting position.

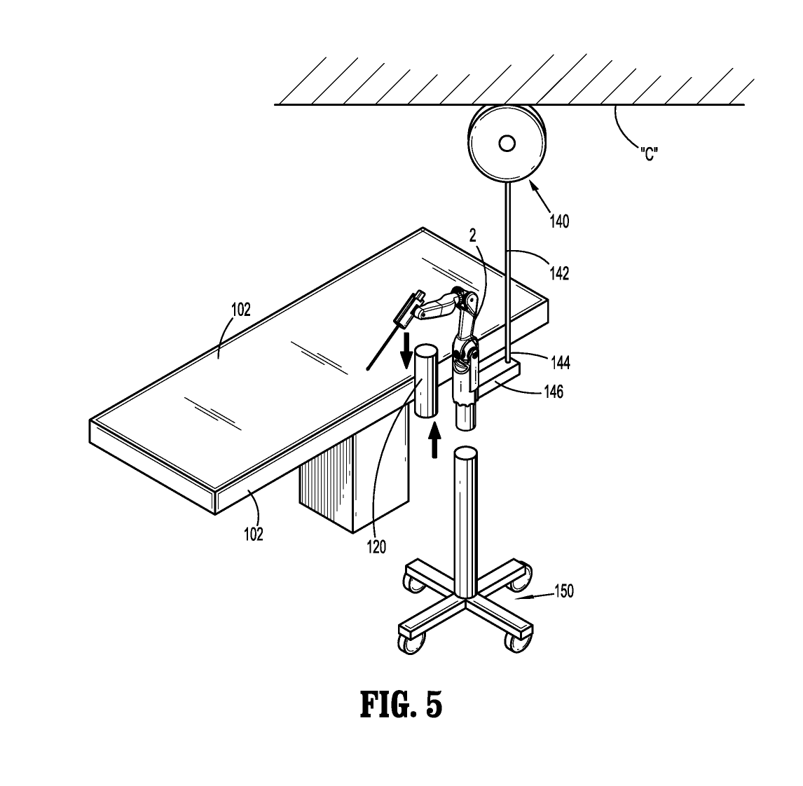

In another embodiment, as shown in FIG. 5, surgical robotic arm support system 100 includes a spool 140 configured to be coupled to a fixed surface, e.g., a ceiling “C” of a surgical operating room. Spool 140 includes a retractable tether 142 having an end 144 configured to be coupled to surgical robotic arm 2. In embodiments, end 144 of tether 142 has a securement device 146 that releasably secures to one of surgical robotic arms 2, 3. In embodiments, spool 140 may include a motor (not shown) that drives movement of tether 142 relative to ceiling “C.” In some embodiments, spool 140 may be operated as a pulley to move tether 142 relative to ceiling “C.” Surgical robotic arm support system 100 may further include a portable base 150 configured to support, move, and/or transport surgical robotic arms 2, 3.

In operation, surgical robotic arm 2, 3 may be coupled to portable base 150 and transported to a position adjacent surgical table 102, as shown in FIG. 5. End 144 of tether 142 is attached to surgical robotic arm 2 and spool 140 is actuated to raise surgical robotic arm 2 to the selected mounting position 112a, 112b, 112c, or 112d on surgical table 102 and then lowered into channel 122 of mounting member 120.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic arm support system, comprising:
a rail configured to be coupled to a surgical table and including a plurality of discrete robotic arm mounting positions; and
at least one mounting member configured to be removably coupled to the rail at a selected one of the plurality of discrete robotic arm mounting positions, wherein each mounting member is configured to support a surgical robotic arm.

2. The surgical robotic arm support system according to claim 1, wherein each of the plurality of discrete robotic arm mounting positions has discrete indicia associated therewith.

3. The surgical robotic arm support system according to claim 2, further comprising a processor configured to:
identify a target mounting position for each surgical robotic arm relative to the surgical table; and
output the identified target mounting position of each surgical robotic arm by displaying the discrete indicia corresponding to the identified target mounting position on a display.

4. The surgical robotic arm support system according to claim 1, wherein each mounting member includes a channel configured for slidable receipt of a respective surgical robotic arm.

5. The surgical robotic arm support system according to claim 1, wherein the at least one mounting member is configured to be slidingly coupled to the rail.

6. The surgical robotic arm support system according to claim 1, further comprising a spool configured to be coupled to a fixed surface, the spool including a retractable tether having an end configured to be attached to the surgical robotic arm.

7. The surgical robotic arm support system according to claim 1, wherein the surgical table has a short side and a long side, wherein the rail is mounted to the long side of the surgical table.

8. A surgical robotic arm support system, comprising:
a surgical table for supporting a patient thereon and including a plurality of discrete robotic arm mounting positions; and
at least one mounting member configured to be fixedly coupled to the surgical table at only a selected one of the plurality of discrete robotic arm mounting positions, wherein each mounting member is configured to support a surgical robotic arm.

9. The surgical robotic arm support system according to claim 8, wherein each of the plurality of discrete robotic arm mounting positions has discrete indicia associated therewith.

10. The surgical robotic arm support system according to claim 9, further comprising a processor configured to:
identify a target mounting position for each surgical robotic arm relative to the surgical table; and
output the identified target mounting position of each surgical robotic arm by displaying the discrete indicia corresponding to the identified target mounting position on a display.

11. The surgical robotic arm support system according to claim 10, wherein each mounting member includes a channel configured for slidable receipt of a respective surgical robotic arm.

12. The surgical robotic arm support system according to claim 10, wherein the at least one mounting member is configured to be slidingly coupled to the surgical table.

13. The surgical robotic arm support system according to claim 10, further comprising a spool configured to be coupled to a fixed surface, the spool including a retractable tether having an end configured to be attached to the surgical robotic arm.

14. The surgical robotic arm support system according to claim 10, wherein the surgical table has a short side and a long side, wherein a rail is mounted to the long side of the surgical table.

15. A method of mounting surgical robotic arms to a surgical table, comprising:
identifying a plurality of discrete mounting positions for surgical robotic arms on a surgical table and a location of each mounting position;
determining an optimal number of surgical robotic arms for a selected surgical procedure;
calculating distances from the discrete mounting positions to a surgical site access point on the patient based on the mounting position locations and at least one of a patient dimension and a placement position of the patient on the surgical table;
identifying an optimal mounting position for each of the optimal number of the surgical robotic arms for the selected surgical procedure from the plurality of discrete mounting positions based on the calculated distances; and
outputting the identified optimal mounting positions.

16. The method according to claim 15, further comprising:
receiving a patent placement signal from a sensor detecting a placement of the patient on the surgical table;
calculating distances from the discrete mounting positions to the surgical site access point based on the placement of the patient on the surgical table according to the patient placement signal.

17. The method according to claim 15, further comprising:
receiving patient weight or height data from a respective surgical table sensor;
calculating distances from the discrete mounting positions to the surgical site access point based on the patient weight or height data.

18. The method according to claim 15, further comprising automatically moving a surgical robotic arm along a rail of the surgical table to its identified optimal mounting position.

19. The method according to claim 15, further comprising:
obtaining surgery preference data associated with the selected surgical procedure; and
identifying from the preference data a subset of instruments used during the selected surgical procedure; and
identifying the optimal mounting position for each of the optimal number of the surgical robotic arms for the selected surgical procedure that maximize a maneuverability of the subset of instruments at a surgical site.

20. The method according to claim 19, further comprising:
identifying a specific surgical robotic arm to which at least one of the subset of instruments should be attached to maximize maneuverability of the at least one instrument at the surgical site; and
outputting an identifier of the identified specific surgical robotic arm.

21. The method according to claim 19, further comprising determining that the optimal number of surgical robotic arms for the selected surgical procedure is a number of preferred robotic arms indicated in the obtained surgery preference data when the obtained surgery preference data includes the number of the preferred robotic arms.

22. The method according to claim 15, further comprising:
calculating distances from the discrete mounting positions to a surgical site access point on the patient based on a dimension of the surgical robotic arms, a dimension of the patient, a dimension of the surgical table, and a distance of a target tissue area to a surgical instrument affixed to at least one of the surgical robotic arms.
